(19) United States
(12) Patent Application Publication
(10) Pub. No.: US 2014/0004505 A1
(43) Pub. Date: Jan. 2, 2014

(54) CARTRIDGE BASED SYSTEM AND METHOD FOR DETECTING AN ANALYTE IN A SAMPLE

(71) Applicants: Xiaoli Su, Fayetteville, AR (US); Steve Charles Green, West Fork, AR (US); Robert Calvin Goforth, Fayetteville, AR (US); Qian Sun, Fayetteville, AR (US); James Randall Andress, Fayetteville, AR (US); Katherine L. Hall, Elkins, AR (US); Chien V. Nguyen, Fayetteville, AR (US)

(72) Inventors: Xiaoli Su, Fayetteville, AR (US); Steve Charles Green, West Fork, AR (US); Robert Calvin Goforth, Fayetteville, AR (US); Qian Sun, Fayetteville, AR (US); James Randall Andress, Fayetteville, AR (US); Katherine L. Hall, Elkins, AR (US); Chien V. Nguyen, Fayetteville, AR (US)

(73) Assignee: BIODETECTION INSTRUMENTS, LLC, Fayetteville, AR (US)

(21) Appl. No.: 14/004,299

(22) PCT Filed: Mar. 9, 2012
(86) PCT No.: PCT/US2012/028449

§ 371 Date: Sep. 10, 2013

Related U.S. Application Data
(60) Provisional application No. 61/451,402, filed on Mar. 10, 2011.

(51) Int. Cl. G01N 33/53

(52) U.S. Cl. CPC G01N 33/5304
USPC 435/5; 422/501; 435/283.1; 422/69; 436/501; 435/7.37; 435/7.2

(57) ABSTRACT

The invention pertains to a disposable cartridge suitable for use in detecting the presence of analytes in fluid samples. The cartridge is designed to provide “lab on a chip” capabilities and comprises a plurality of sample storage reservoirs and analytical compartments/channels. A valve system based on valve disks aids in securely transferring samples from storage reservoirs to analytical channels and detectors with minimal use of common channels, which reduces risk of cross-contamination.

### CROSS REFERENCE TO RELATED APPLICATION

This application claims priority from U.S. Provisional Application 61/451,402 filed on Mar. 10, 2011, which is incorporated by reference in its entirety.

### BACKGROUND OF THE INVENTION

The present invention pertains to the field of analyte detection. More specifically, the present invention generally relates to a system and method for detecting analytes in a fluid. In particular, the invention relates to a cartridge-based system for detecting and/or obtaining analytical measurements of an analyte in a sample using a semi-enclosed microfluidics network and automated detection and quantification.

Processes and equipment for detecting the presence of an analyte in a sample are critical to several technology areas ranging from food preparation to homeland security to trauma care. For example, contamination of food products by pathogenic bacteria, due to human negligence or bioterror attack, poses a tremendous threat to the public. Rapid detection of foodborne pathogens is a critical step to maintaining food safety and security.

Current methods used to detect contaminants (e.g., microorganisms, toxins, and pesticides) and/or obtain quantitative data of contaminant levels typically are cumbersome, expensive, and involve considerable time delay. Many of the known analytical systems for accomplishing such tasks are large and expensive; requiring specialized training and laboratory controls. In addition, many available systems are dedicated to identifying and/or quantifying only one or a few contaminants. Of particular importance is the long delay time, ranging from hours to days, required to obtain analytical information, particularly of biological contamination.

Timely identification of contaminants is of utmost importance. For example, in a food preparation processes, there is an urgent requirement to demonstrate that bacteria concentration is below a threshold level at the beginning and ending of a processing run. Current testing protocols can result in considerable downtime for a food processing line due to the time lag in obtaining test results.

There are many other applications that can benefit from an analytical system having lower cost, less complexity, and faster response time. Besides food safety, applications may include homeland defense, military, environmental monitoring, chemical processing, and medical or clinical diagnostics.

In the past several years researchers, particularly in the medical arena, have expended considerable time and effort in developing point-of-care analytical devices that can greatly accelerate the process of sample preparation and analyte detection. Such devices are often broadly referred to as “labs-on-a-chip” and their construction, field of use, and cost can vary widely. Exemplary devices that would fall within this category are discussed in U.S. Pat. No. 8,105,849 and US Patent Application Publication 2011/0104024.

One particular diagnostic technology that is suited for potential use in a “lab-on-a-chip” is the immunoassay. Immunoassays are useful for the detection of pesticide residues, toxins, protein biomarkers, pathogenic bacteria, and many other substances. Immunoassays, particularly the enzyme-linked immunosorbent assay (ELISA), have been widely used as alternatives to the time-consuming culture-plating method for rapid microbial detection owing to their high specificity and versatility. Yet many current immunoassay based tests fail to provide the versatility need for many applications. Compact, one step, immunoassay tests are often limited to detection of one or two analytes (e.g., the common canine heartworm test) and only provide a “yes/no” result based on a manual reading of a colorimetric change for a single sample. Many of the heterogeneous immunoassays use microwell plates, membranes, or beads as solid supports for immobilizing specific antibodies or antigens. However, these methods typically involve multi-step manual liquid handling and are thus laborious and tedious.

More recent research has introduced capillary columns and microfluidic chips into immunoassay technology. Capillary columns and microfluidic chips are advantageous because the flow-through geometry is more convenient for liquid handling. In addition, such columns and chips are compatible with automation, which is critical in the heterogeneous immunoassays that involve multiple steps of reagent addition and washing. Another advantage is that the consumption of immunoreagents, usually scarce and expensive, can be reduced because of the small dimensions of capillaries. Moreover, capillary geometry enhances assay kinetics due to the high surface-area-to-volume ratio and restricted diffusion of reacting agents. Unfortunately, adoption of capillary columns and microfluidic chips into bioassay technology has been slowed by cross-contamination risks when running multiple samples through a system. Typically, engineers address this problem by creating complex valve systems to control sample flow and post-sample cleaning of capillary channels and tubes. Such systems tend to be bulky and expensive. Since they are expensive they are not well suited for use in a disposable cartridge that is designed for rapid and reliable screenings.

There is a need for a rapid, accurate, flexible, and cost-effective system to detect the presence of contaminants in samples, particularly biological contaminants. Such a system should also provide quantitative data related to the concentration of the contaminants if they are present. Such a system should be easy to use and its operation should not require extensive training of personnel. Preferably, such a system would be based on a disposable cartridge that is designed for cost-effective mass production and automation analysis of samples.

### SUMMARY OF THE INVENTION

The claimed invention encompasses a disk for controlling the flow of fluid through a cartridge housing. The disk comprises a circular first face having a first channel the first channel is suitable for establishing fluid communication between a first fluid portal and a second fluid portal of the cartridge housing.

The claimed invention also encompasses a cartridge for use in detecting an analyte in a fluid sample where the cartridge comprises a cartridge housing; a reservoir integral with the cartridge housing; a connecting channel integral with the cartridge housing; a first valve capable of establishing fluid communication between the reservoir and the connecting channel; a reaction channel having an inlet and an outlet, the reaction channel integral with the cartridge housing; a detection channel integral with said cartridge housing; and a second valve capable of establishing fluid communication (1) between the connecting channel and the reaction channel, and (2) between said reaction channel and said detection channel.

In addition, the invention also provides a method for detecting an analyte in a fluid sample, the method comprising the steps of placing a sample in a first reservoir; aligning a first valve to establish fluid communication between said reservoir and a connecting channel; aligning a second valve to establish fluid communication between said connecting channel and the inlet of a reaction channel and the outlet of said reaction channel and an analyte detection channel to thereby establish fluid communication between said reservoir and said analyte detection channel; drawing a sample from said first reservoir through said reaction channel and through said detection channel; and detecting the presence of an analyte in the sample as said sample passes through said analyte detection channel.

### BRIEF DESCRIPTION OF THE DRAWINGS

FIG. 1 is a perspective exploded view of a cartridge according to the invention and includes a drive mechanism.

FIG. 2A is a view in which a cartridge housing is presented in a transparent form.

FIG. 2B is an enlarged view of a first valve as shown in FIG. 2A.

FIG. 2C is an enlarged view of a second valve as shown in FIG. 2A.

FIG. 3 is a transparent view of the bottom surface of a cartridge housing.

FIG. 4A is a perspective view of a valve disk and a disk support.

FIG. 4B is a perspective view of one end of a disk support.

FIG. 4C is an exploded view illustrating a biasing mechanism.

FIG. 5 is a schematic view of possible fluid flow through a cartridge according to the invention.

FIG. 6 is a schematic view of the various components to a system for detecting analytes in a fluid.

FIG. 7 is a schematic illustrating a cross-section of a reaction channel.

FIG. 8 illustrates a drive mechanism engaging with a cartridge according to the invention.

FIG. 9 illustrates a commercial embodiment of a system according to the invention.

FIG. 10 provides sample analytical data provided by the invention.

FIG. 11 is a view of the top surface of a cartridge housing.

### DETAILED DESCRIPTION

In the following description, for purposes of explanation, numerous details are set forth to provide an understanding of one or more embodiments of the present invention. Furthermore, the following detailed description is of the best presently contemplated mode of carrying out the claimed invention based upon existing experimental data. The description is not intended in a limiting sense, and is made solely for the purpose of illustrating the general principles of the invention.

Furthermore, since one aspect of the invention is a mechanical device, the device according to the invention is described in relation to the architecture of the exemplary embodiments shown in the Figures. Those skilled in the art will recognize that the arrangement of components shown in the Figures and discussed herein has certain advantages over known “lab-on-a-chip” devices such as reduction of the length of common channels, ease of manufacture, etc. However, the geometrical arrangements of the various components discussed herein should not be interpreted as limiting the scope of the invention, which is only limited by the claims.

Directional terms such as “top” and “bottom” are used as a narrative convenience to illustrate relative positioning of various elements and the most likely orientation of a device during operation. The devices discussed herein are capable of working in several different spatial orientations (e.g., at an angle, etc.).

The term “diameter” as used herein should not be interpreted in a strictly geometrical since as the length of a straight line through the center of a circle. The term “diameter” as used herein is meant to convey the general concept of cross-sectional size of a capillary channel or tube-like structure. In some instances these channels may be cylindrical and in some instances they may resemble an enclosed “U” or even a rectangle. Thus the term “diameter” is to be viewed as giving relative cross-sectional scale to any particular element described herein.

As used herein the term analyte includes any substance that is capable of detection. For example, the term analyte includes biological compounds, inorganic compounds, elements of the periodic table, and organic compounds, and combinations of any and all of these. In particular, the term analyte is meant to include contaminants such as pesticides, herbicides, chemicals, pharmaceuticals, biological fluids (e.g., blood, urine, saliva, cerebrospinal fluid, breast milk, sweat, tears,), microbes (e.g., viruses, bacteria, fungi, protozoans), and combinations of any and all of these. More specifically, the term analyte includes substances such as nucleotides, nucleic acids (e.g., RNA, DNA, mRNA, miRNA, rRNA), proteins, peptides, amino acids, antibodies, antigens, haptens, monosaccharides, polysaccharides, lipids, elements of the periodic table, inorganic compounds, organic compounds, and combinations of any and all of these.

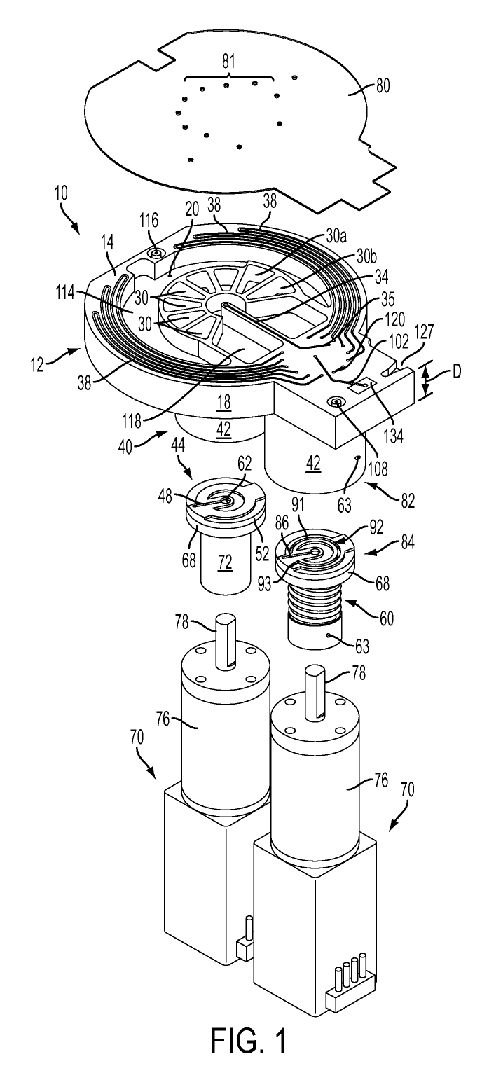

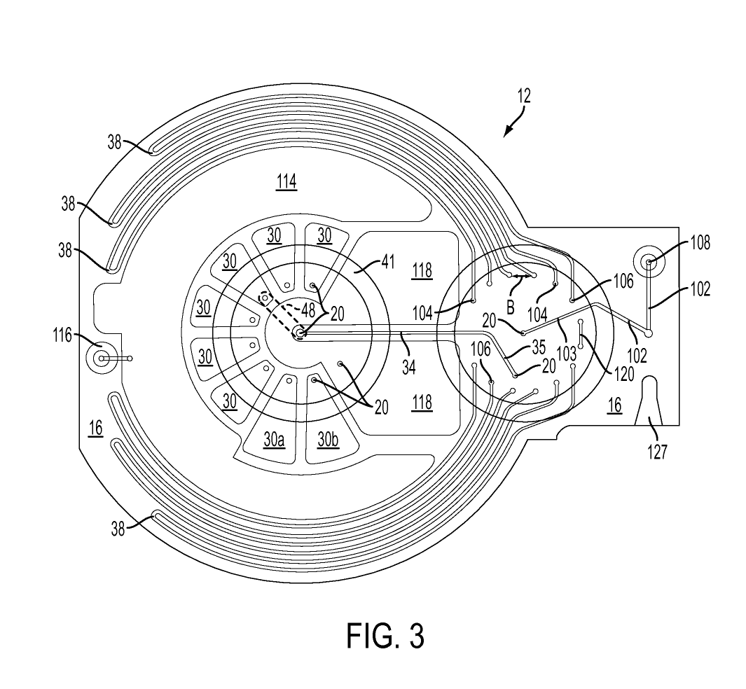

Turning now to FIGS. 1 and 3, one aspect of the invention is a cartridge 10 that is suitable for use in the rapid detection and/or quantification of an analyte in a sample. In preferred embodiments, the cartridge 10 comprises a cartridge housing 12, a first reservoir 30, a connecting channel 34, a first valve 40 capable of establishing fluid communication between the first reservoir 30 and the connecting channel 34, a first reaction channel 38 having an inlet 104 and an outlet 106, a detection channel 102, and a second valve 82 capable of establishing fluid communication (1) between the connecting channel 34 and a first reaction channel 38, and (2) between a first reaction channel 38 and the detection channel 102. The reservoir, connecting channel, reaction channel, and detection channel are all integral with the cartridge housing. Preferably the cartridge according to the invention contains a plurality of recesses and channels that are integral with the cartridge housing and are capable of being placed in fluid communication with each other via the valves as discussed below.

Generally speaking, most commercial embodiments of the cartridge 10 according to the invention can be generally divided into a storage portion and an analytical/detection portion. The storage portion of the cartridge is roughly that part that houses the reservoirs 30, a wash reservoir 118 (discussed below), the first valve 40, and the entrance to the connecting channel 34. The analytical/detecting portion of the cartridge can be considered to be the part the houses the terminal end of the connecting channel 34, the second valve 42, the reaction channels 38, and the detection channel 102. Each of these elements and others are discussed in more detail below.

Beginning the with cartridge housing 12, the cartridge housing 12 is preferably manufactured from a single block of material and is defined by a first or “top” surface 14 and a second or “bottom” surface 16 spaced apart therefrom thereby defining a housing body 18. The cartridge housing body 18 is defined by a width “D” separating the top surface 14 and the bottom surface 16.

Preferably, the cartridge housing 12 is manufactured from a material that is suitable for low cost yet precision manufacture. In preferred embodiments the cartridge housing 12 is made from a polymer possessing the relevant physical characteristics (e.g., chemical inertness) suitable for use in detecting the analyte at issue. An exemplary polymer suitable for use in manufacturing cartridge housings 12 for many biological applications is a polycarbonate polymer such as Lexan, which is commercially available from SABIC Innovative Plastics. It is understood that other polymers may be suitable for use in forming the cartridge housing 12 if they possess the requisite

In its most basic form, the cartridge housing 12 according to the invention contains at least one reservoir 30 suitable for receiving and retaining a liquid sample, a connecting channel 34, and a reaction channel 38. In preferred embodiments the housing 12 contains a plurality of reservoirs 30 (e.g., a first reservoir, a second reservoir, a third reservoir, etc.) and a plurality of reaction channels 38 that are connected to the connecting channel 34 by one or more valves.

Focusing now on the reservoirs 30, the reservoirs 30 are integral with the cartridge housing 12 in that they are formed from recesses in the cartridge body 18. In the embodiment shown in FIGS. 1 and 3, the reservoir 30 is a triangular shaped recess generally located in the central portion of the cartridge housing 12. The reservoir 30 can be formed either by milling the cartridge housing 12 or by molding the housing to incorporate the recess. As shown in FIG. 3, in preferred embodiments the cartridge housing 12 contains a plurality of reservoirs arranged in an arcuate fashion in the central portion of the housing 12, each suitable for receiving a liquid sample containing an analyte.

The exact size, shape, volume and arrangement of the reservoirs 30 are variable and may be adjusted depending upon the needs of the particular application. For example, the testing technology for one particular analyte may require several milliliters of a sample while another may require only several hundred microliters. Accordingly, those skilled in the art can vary the size and shape of the reservoirs to meet their particular needs.

In some instances the testing technology incorporated into the practice of the invention (e.g., immunoassay technology) may require that a reagent be introduced into the reaction channel 38 after a sample prior to testing. In this event one or more of the plurality of reservoirs 30 can be utilized to hold a reagent instead of a sample. In the embodiment shown in FIGS. 1 and 3, two of the reservoirs 30 are shown to be slightly larger than the others and are designated as 30a and 30b. They are larger than the others because for cartridges capable of handling multiple samples one typically needs a sufficient amount of reagent for testing each sample.

One important aspect of the device according to the invention is the efficient transfer of one or more fluid samples from the sample storage portion of the cartridge housing 12 to the analytical/detecting portions of the cartridge housing 12. This aspect of the invention is accomplished by use of a novel and efficient valve and channel system. In a sense, the valve and channel system begins with the reservoirs 30 because fluid samples must leave the sample reservoirs to be analyzed. Fluid samples leave individual reservoirs 30 via fluid portals 20. Each reservoir 30 is in direct fluid communication with a fluid portal 20.

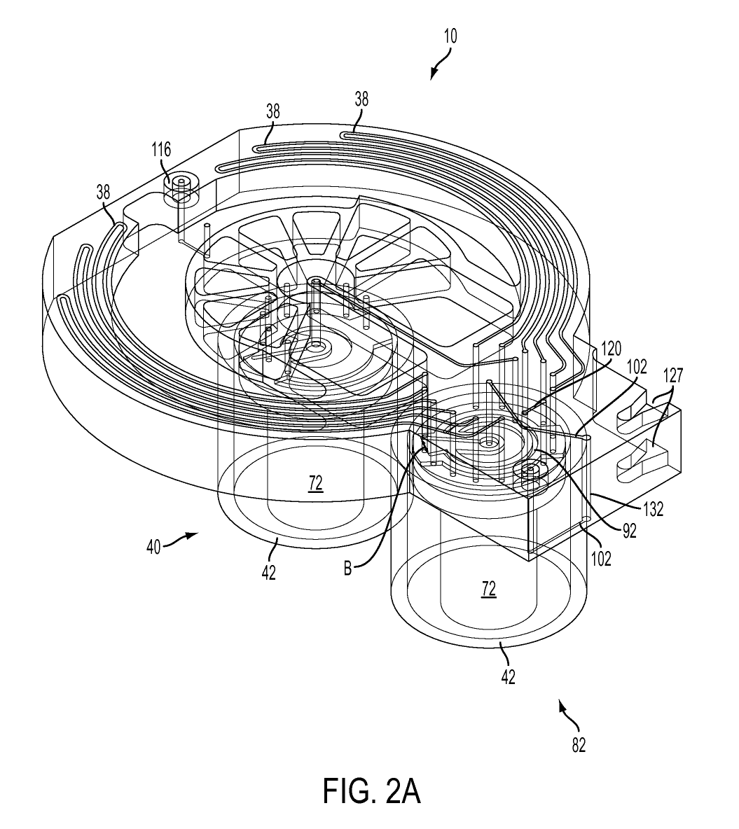

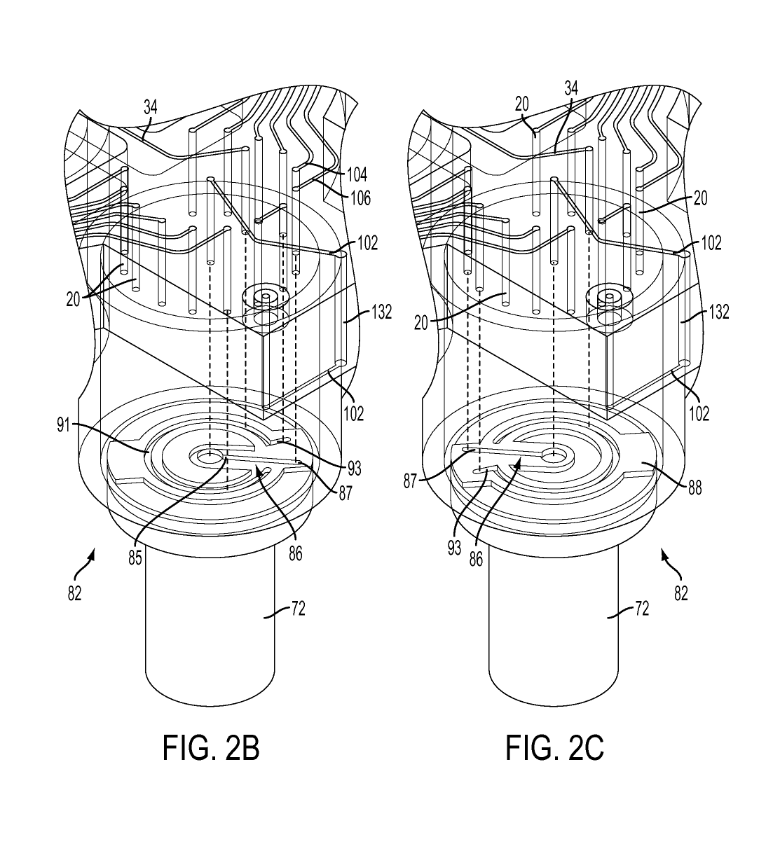

The fluid portals 20 serve as a means to facilitate the transfer of fluid from one part of the cartridge housing 12 to another part of the housing. In most instances a fluid portal 20 is a vertical capillary channel or tube that establishes fluid communication between the top surface 14 of a cartridge and the bottom surface 16 of a cartridge. Such portals are best seen in the transparent view of the housing shown in FIGS. 2A, 2B, and 2C. In other views, such as FIG. 3, they are represented by small holes (circles). Typically, at least one fluid portal is associated with each reservoir or channel. Therefore it should be understood that when a channel or reservoir is said to be in fluid communication with each other there is typically a fluid portal 20 that marks the termination of one channel and the start of another.

In addition, and very generally speaking, the flow of fluid through the cartridge follows a repeating pattern. Fluid flows from a recess in the top surface 14 of the cartridge (e.g., a reservoir) through a fluid portal to a valve on the bottom surface 16 of the cartridge. The valve transfers the fluid via another fluid portal back to the top surface 14 where it enters another channel. The fluid runs the length of that channel, then goes back down through the cartridge (via a fluid portal) to a second valve where it is selectively distributed to another top surface channel (via a fluid portal). The fluid may make one or two more “top to bottom” transfers but eventually makes its way past a detector and waste collector. In short (and in simplistic terms), fluid is stored and moved between top surface compartments via valves that are on the bottom surface. The following paragraphs will expand upon this general framework of fluid flow.

In the embodiment of the invention shown in FIG. 3, each reservoir 30 has a fluid portal 20 that establishes fluid communication through the body 18 of the cartridge housing 12. The portals 20 may be formed in the housing 12 by any acceptable means such as milling/drilling or molding. Typically, the diameters of the fluid portals 20 are the same or close to the diameters of the various channels.

The fluid portals 20 provide the means for transferring sample fluid from the reservoirs 30 to the valve system. In preferred embodiments the cartridge 10 according to the invention is capable of analyzing multiple samples and analytes. Thus, and as set forth in greater detail below, the preferred embodiments of the cartridge 10 comprise two valve systems that are very similar in form and function. For purposes of this detailed description, valve components that are substantially similar in each valve will be identified using the same reference numbers. Those components that are substantially different will be identified using different numbers. In addition, the figures will aid the reader in understanding the similarities and differences between individual valve components.

We begin the description of the valve system with a discussion of the first valve 40 and how it facilitates transfer of sample fluid from one or more reservoirs 30 to a connecting channel 34.

As shown in FIG. 1, the first valve 40 comprises a valve housing 42. In preferred embodiments the valve housing 42 is cylindrical. The valve housing 42 attaches to the bottom surface 16 of the cartridge housing 12 such that the fluid portals 20 that connect to the reservoirs 30 are encompassed within the area enclosed by the valve housing 42. In other words, the fluid portals 20 are in fluid communication with the lumen of the valve housing 42. Also, it should be noted that in the embodiments incorporating multiple reservoirs 30 the fluid portals are arranged in a generally circular fashion. The significance of this arrangement is shown below.

Preferably, the valve housing 42 is made from the same material as the cartridge housing 12 but it can be made of a different material if needed. It is securely attached to the bottom surface 16 of the cartridge housing 12 by any suitable means. For example, a cylindrical recess 41 can be milled or molded in the bottom surface 16 to tightly receive the valve housing 42 as shown in FIG. 3. A suitable sealer/bonding agent can then be used to attach the two components. Alternatively, the valve housing 42 may simply be glued or otherwise bonded (e.g., heat bonded) to the bottom surface 16 of the cartridge 10. A further alternative is to cast or mold the valve housing 42 and cartridge 12 housing as one integral unit.

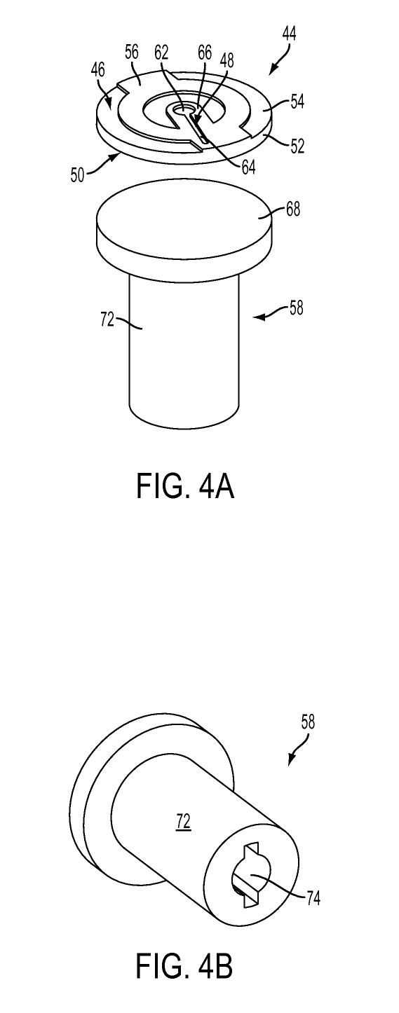

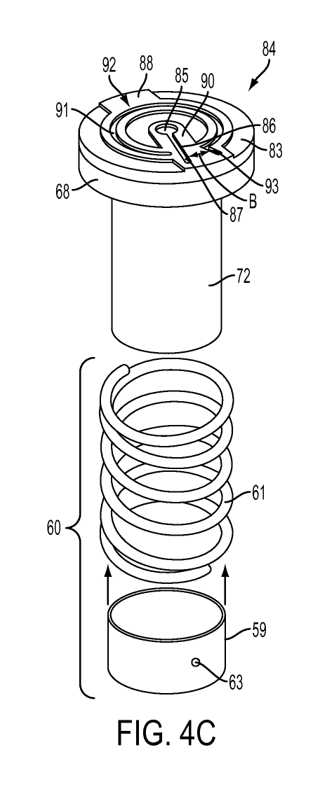

The valve housing 42 receives multiple components that form the overall valve 40. These components are generally shown in FIGS. 1 and 4A-C. One such component is the first valve disk 44 that is supported by a valve support 58. The valve 40 also comprises a biasing mechanism 60 that provides continuous fluid tight contact between the first valve disk 44 and the bottom surface of the valve housing 42. Each component is discussed in turn.

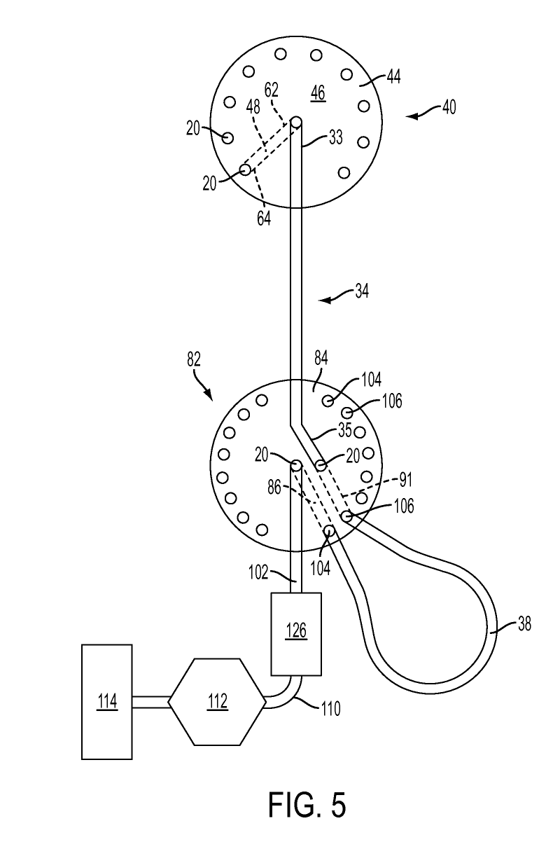

Turning now to FIGS. 1 and 4A, the flow of fluid through the valve 40 is controlled in large part by the first valve disk 44. The first valve disk 44 is defined in part by a circular first face 46 and a circular second face 50 separated from the circular first face 46 to thereby define a disk body 52. A first channel 48 resides on the circular first face 46. The first channel 48 is suitable for establishing fluid communication between a first reservoir 30 and a connecting channel 34 of the cartridge housing 12. When there are multiple reservoirs 30 in the storage portion of the cartridge housing 12 the valve disk 44 and first channel 48 are capable of providing selective fluid communication between the individual reservoirs 30 and the connecting channel 34. As used herein the term “selective communication” means that the valve disk 44 is capable of providing sequential and discreet fluid communication between one or more reservoirs 30 and a connecting channel 34. FIG. 5 provides a schematic representation of the relationship between the first channel 48 and the reservoirs/fluid portals.

This capability to provide selective fluid communication is critical for embodiments of the inventive cartridge 10 that analyze multiple samples. The ability to provide fluid communication between one reservoir 30 and the connecting channel 34 while preventing fluid communication between other reservoirs and the connecting channel 34 helps avoid cross-contamination of samples and greatly facilitates the flushing/washing functions of the cartridge which further avoids cross-contamination. The flushing/washing capabilities of the inventive cartridge are discussed in more detail below.

The first channel 48 is defined by a first end 62 and a second end 64 separated by an intermediate portion. The length of the first channel 48 is sufficient to create fluid communication between the connecting channel 34 of the cartridge housing 12 (specifically the fluid portal 20 that marks the entrance to the connecting channel 34) and the fluid portal or portals 20 that are connected to the reservoir or reservoirs 30. The first end 62 of the first channel 48 encompasses the center of the circular first face 46 where it is in fluid communication with the connecting channel 34. The second end 64 is at a point distal to the center of the circular first face 46 where it will establish fluid communication with one or more fluid portals 20 that are integrated into a reservoir or reservoirs 30.

It should be readily apparent to one skilled in the art that the valve 40 is designed so that by rotating the first valve disk 44 the second end 64 of the first channel 48 moves in an arc that brings it into fluid communication with each reservoir 30 (via fluid portals 20). It should also be readily apparent that fluid communication between a reservoir 30 and the first channel 48 necessarily establishes fluid communication between the reservoir 30 and the connecting channel 34. FIG. 5 schematically illustrates the mechanical relationship between the rotation of the first channel 48 and the selective fluid communication between the reservoirs 30 (represented by their fluid portals 20) and the connecting channel 34.

As discussed above, it is possible to form a valve disk 44 in which the first channel 48 is located directly on the circular first face 46. Such an arrangement typically requires creating an extremely flat first face 46 and an extremely flat bottom housing surface 16 to facilitate a fluid tight seal between the two. Such precision is often unattainable on a consistent basis, which can result in an unacceptable level of product failure. In addition, efforts to attain such precision increase manufacturing costs.

Accordingly, in a more preferred embodiment of the first valve disk 44 the circular first face 46 is further defined by a first surface 54 and a second surface 56. The second surface 56 is spaced apart from and is generally parallel to the first surface 54 such that the second surface 56 forms a raised platform on the first surface 54. This arrangement, in combination with preferred materials of construction, provides the ability to create a fluid channel 48 that is in a moveable, fluid-tight arrangement with the cartridge housing 12 without the use lubricants and/or sealers.

The phrase “fluid tight” as used herein means a structural relationship between two parts of the invention that prevents the unwanted leakage or transfer of fluid outside of designated channels.

The shape and geometry of the second surface 56, while not critical to the practice of the invention, can aid in the overall performance of the first valve disk 44 and the valve 40. In a preferred embodiment of the invention shown in FIG. 4A, the second surface 56 is generally annular, concentric with the center of the first valve disk 44 and has an outer diameter that is less than the diameter of the diameter of the first valve disk 44. This arrangement results in a raised ring slightly inside the edge of the first valve disk 44.

The second surface 56 also possesses an arm 66 extending radially from the center of the disk first face 46 to its outer edge. In preferred embodiments the first channel 48 is located within this arm 66 of the second surface 56. Placing the first channel 48 within this arm 66 enables one to provide suitable depth to the channel while simultaneously surrounding the channel with a pliable substance that aids in forming a fluid tight seal with the cartridge housing 12. The same principle applies to the raised annular ring formed by the second surface 56. The pressure applied by the biasing mechanism 60 (discussed below) causes the pliable yet low friction polymer that forms the annular second surface 56 to seal the interior of the valve 40 thus preventing contamination of samples and leakage.

With respect to materials of construction, in preferred embodiments the valve disk 44 is formed of a polymer having a coefficient of friction that is sufficient to allow lubricant free rotation against the cartridge housing 12. Generally speaking, it is envisioned that in the majority of circumstances the valve disk 44 will be formed of a polymer material having a coefficient of friction and a hardness that is less than that of the polymer utilized in making the cartridge housing 12.

In addition, it is envisioned that the first valve disk 44 will be formed of a polymer that has a degree of compliance or deformability sufficient create a fluid tight seal with the cartridge housing.

Such polymers include polyolefins. Polyolefins are a preferred class of polymers due to their commercial availability, relative chemical inertness and physical properties that are well suited for mass production. For example, a polyolefinic block copolymer manufactured by RTP Co. and having a specific gravity of 0.89, a tensile strength of 7 MPa, a tensile elongation of >500%, tensile stress of 3.1 MPa, tear strength of 49.1 N/mm, hardness (Shore A) of 80, melt temperature of 182-227° C., and mold temperature of 21-79° C. worked well with respect to forming the first valve disk 44 and the operation of the disk. Another benefit of polyolefins is that they can be modified to possess physical characteristics required by a particular application. Other polymers with similar characteristics are also suitable for use in the practice of the invention.

The first valve disk 44 is supported by a disk support 58. The disk support 58 may be made from any substance having the necessary strength to perform its function, which is to support the first valve disk 44, engage with a drive mechanism 70, and thereby rotate the first valve disk 44. In preferred embodiments the disk support 58 is made of a polymer to reduce manufacturing costs. The disk support 58 may be made of the same polymer as the valve disk 44 or a different polymer. In most instances it is envisioned that the disk support will be made out of a harder, stronger polymer than the disk valve 44 to increase its functional life. However, if a polymer with the strength, compliance, and friction characteristics suitable for performing all of the functions of the valve disk 44 and the disk support 58 is developed, then the valve disk 44 and the disk support 58 may be formed as a single integrated part.

The disk support 58 is comprised of two parts: a support platform 68 and a support spindle 72. The support platform 68 is generally a solid disk of the same diameter as the first valve disk 44 and is fixably attached to the first valve disk 44 second face 50. The valve disk 44 may be attached to the support platform 68 using any suitable means such as thermal bonding, adhesives, etc. If the valve disk 44 and the disk support 58 (and/or support platform 68) are formed of two different polymers the use of an overmolding technique to form a unified valve disk/support element is a preferred method of attachment. If an overmolding technique is utilized care should be taken to choose two polymers that are compatible with an overmolding technique as not all combinations of polymers can be joined using such a technique.

The support spindle 72 is a generally cylindrical tube having an outer diameter that is less than the outer diameter of the support platform 68. This creates a space between the interior wall of the valve housing 42 and the surface of the spindle 72 in which a biasing mechanism 60 resides. The biasing mechanism 60 is any device capable of forcing the first valve disk 44 up against the bottom surface 16 of the cartridge housing with enough force to create a fluid tight seal between the first channel 48 and the bottom surface 16 of the cartridge housing. In FIGS. 1 and 4C the biasing mechanism is shown as a tension spring 61 held in place by a bushing 59 that is fixed in position within the lumen of the valve housing 42. Prototypes of the cartridge 10 held the bushing in place by pins (not shown) that extended through aligned holes 63 the wall of the valve housing 42. Other types of biasing mechanisms known by those skilled in the art can be used as well (e.g., compressible O-rings instead of springs). The primary factor in choosing the ultimate biasing mechanism 60 is that it performs the required function and is cost effective for a disposable device.

The support spindle 72 is also defined by a drive channel 74 (FIG. 4B) that extends upward from the end opposite the support platform into the interior of the support spindle 72. The drive channel 74 is designed to engage with a drive mechanism 70, specifically a drive shaft 78, as shown in FIG. 1.

In most instances it is envisioned that the drive mechanism 70 will comprise an electric motor 76 and a drive shaft 78 which are controlled by an electronic control system 100 that incorporates a touch pad controller 101. The drive shaft 78 is received by drive channel 74 to provide a means for controlled rotation of the support spindle 72, disk support platform 68, valve disk 44, and first channel 48.

Those skilled in the art recognize that other forms of driving mechanisms may be utilized in the practice of the invention. For example, instead of using a channel/shaft configuration one could use a gearing mechanism where a female gear is molded into the bottom of the support spindle and a male gear is attached to an electric motor.

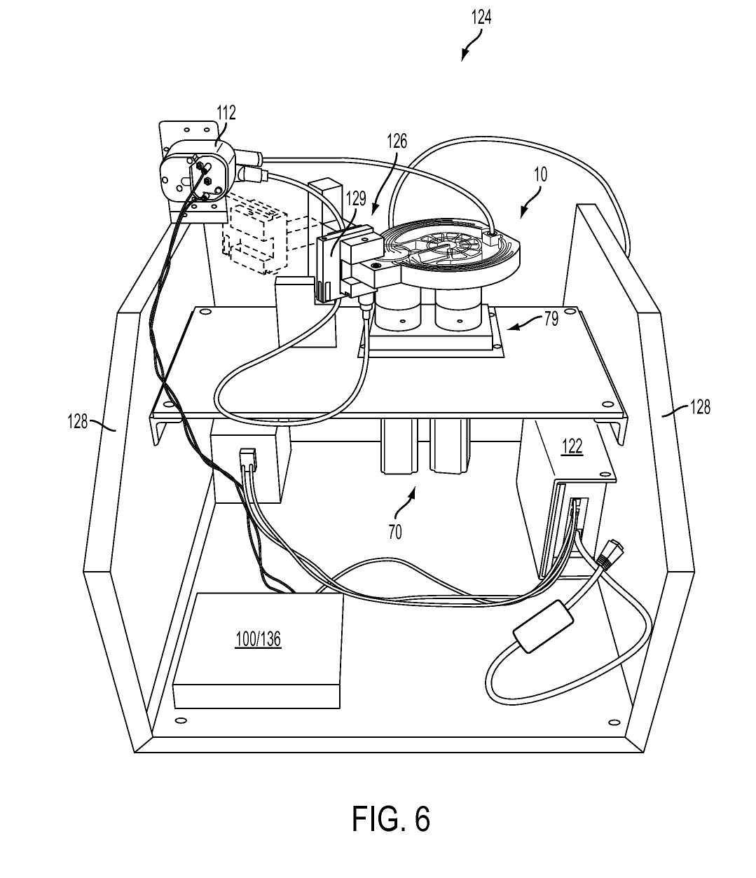

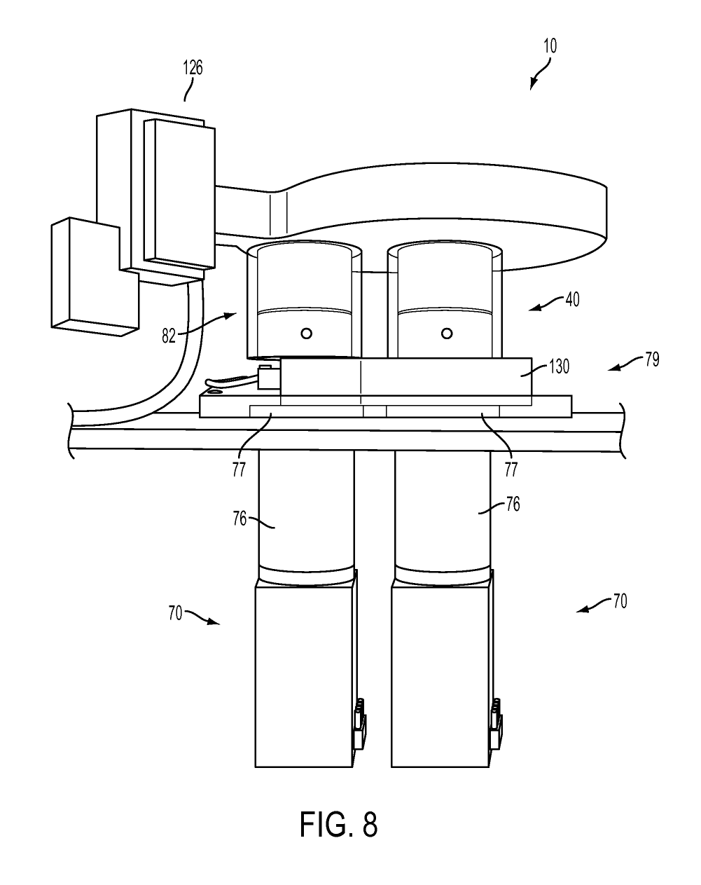

In the embodiments of the invention using two valve systems, such as the embodiment shown in FIG. 1, it was discovered that suspending the motors of the drive mechanism 70 from a supporting frame 79 rather than fixably attaching them to a solid base improved the overall performance of the system over a series of cartridge tests. This arrangement is schematically shown in FIGS. 6 and 8 in which the motors 76 are freely hanging from a frame 79. The means of suspending the motors may vary. For example, the frame 79 could contain two holes having a diameter sufficient to lower the motors down through the holes. Collars 77 (schematically represented in FIG. 8), having a diameter greater than that of the frame holes, could be attached to the bodies of the motor 76. The collars 77 would rest on the frame and the motors 76 would be thus suspended. Guideposts or other suitable means could be placed on the frame to keep the motors 76 from “walking” due to vibratory movement. Other suitable means of suspending the motors can also be employed in the practice of the invention.

The use of suspended motors provides some “give” or “wiggle room” in the mating of the drive shafts 78 and the drive channels 74. This compensates for slight misalignments that might occur in the polymer molding processes and the construction processes that create the assembled valve and cartridge housing 12 combination. For example, because the cartridges 10 are designed for mass production, there will be instances where the drive channels 74 may be slightly off center and not perfectly aligned with the drive shafts 78. In such instances even the slightest misalignment can place a transverse load on the support spindles 72. Transverse loads placed on the support spindles 72 due to misaligned drive channels and drive shafts can compromise the precise fluid transfer provided by the valves 40.

As shown in FIG. 5, in preferred embodiments of the cartridge 10 according to the invention there are a plurality of reservoirs 30 located in the cartridge housing 12. The reservoirs 30, or at least the fluid portals 20 associated with them, are arranged in an arcuate pattern to facilitate selective fluid communication by the valve disk 44. The first valve 40 is positioned such that the first valve disk 44, and more specifically the second end 64 of the first channel 48, establishes fluid communication with each reservoir's fluid portal 20 when the first valve disk 44 (and thus the first channel 48) is rotated in an arc. During operation, the first end 64 of the first channel 48 (the portion that traverses the center of the valve disk 44) is positioned to maintain fluid communication with the fluid portal 20 that marks the entrance of the connecting channel 34. Thus, the first channel 48 is in constant fluid communication with the connecting channel 34. The control mechanism 100 operates the drive mechanism 70 which rotates the first valve disk 44 to sequentially align the second end 64 of the first channel 48 with one or more fluid portals 20 to establish selective fluid communication between the various reservoirs 30 and the connecting channel 34.

The connecting channel 34 is representative of several channels that help define the cartridge housing 12. Accordingly, the description of its function, use, and construction is applicable to the other channels identified herein. The connecting channel 34 is a capillary channel that has portions that reside both within the body 18 of the cartridge housing 12 and on the top surface 14 of the cartridge housing. As used herein the term capillary channel includes channels or tubes that are milled, drilled, molded or otherwise formed into the body and surfaces of a cartridge housing. The size, shape and volume of the capillary channels can vary depending upon the needs of the particular analytical process. For example, it is envisioned that most channels will incorporate a circular or generally semi-circular cross-section depending on whether it is traversing the cartridge body or surface. However, trapezoidal or other cross-sectional shapes may be beneficial in certain circumstances. Generally speaking the channels should be kept as small as possible (in both length and volume) to facilitate the most efficient use of samples, time, materials, and space. Those skilled in the art are capable of optimizing the design parameters (e.g., size, shape, volume, length) that fit their particular needs.

In preferred embodiments, the portions of the capillary channels (fluid portals 20) that traverse the body 18 of the cartridge housing are between 10 μm and 10,000 μm in diameter, more preferably between 100 μm and 1000 μm in diameter, and most preferably between 200 μm and 800 μm in diameter. They can be formed by drilling or molding as known in the art and their cross-sections can vary between circular and square or any other geometric shape desired by the practitioner.

The portions of the capillary channels that run along the top or bottom surfaces of the cartridge housing 12 are formed by milling or molding.

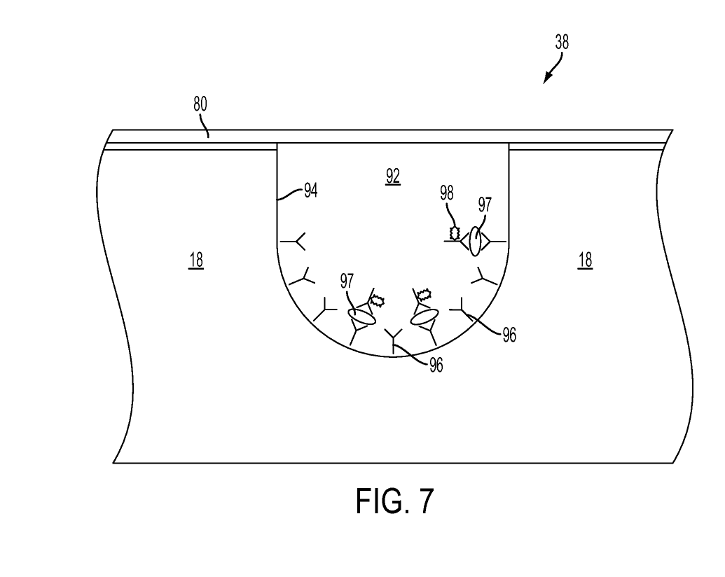

It should be readily apparent that portions of channels such as the connecting channel 34 that run along the surface of the cartridge housing 12 are essentially troughs which are not suitable for pump controlled fluid flow. Therefore, after the channels and recesses are formed in the housing, the top surface 14 of the cartridge housing 12 is covered by a thin polymer sheet 80 which serves to enclose all recesses and channels on the top surface 14 of the cartridge housing 12. To the extent there are channels on the bottom surface 16 of the cartridge, those channels are covered as well by a second polymer sheet. FIG. 7 schematically represents the cross-section of a typical channel (in this case a reaction channel 38) that is formed on the surface of a cartridge housing 12. Again, the exact geometric shape of the channel cross-section can vary depending upon the preferences of the practitioner. Generally speaking, the cross sectional area (and shape) of the surface channel should be similar to that of the fluid portals to promote smooth flow of fluid through the cartridge.

The thin polymer sheet 80 may be any polymer capable of attachment to the cartridge housing 12. In preferred embodiments the polymer sheet 80 is thin and can be punctured at the time of use to provide access to individual reservoirs 30 and facilitate placement of samples therein. Exemplary puncture holes are represented as elements 81 in FIG. 1. It should be noted that this puncturing step means that the cartridge is vented and operates at atmospheric pressure.

The physical properties of the polymer sheet 80 should be such that it does not interfere with the diagnostic purpose of the cartridge. For example, it should not interfere with the processing of the sample (e.g., react with the sample) or possesses optical properties that would hinder the function of an optical detector if an optical detector is used in the practice of the invention. Furthermore, the polymer sheet 80 should be possess the physical characteristics necessary for it to be applied to the housing 12 in a manner such that it does not sag into the surface channels and occlude them.

One such polymer suitable for making the thin polymer sheet 80 is polyethylene terephthalate (PET). This polymer can be applied to a cartridge housing 12 made from a polycarbonate polymer using thermal adhesive processes known to those skilled in the art.

The application of the thin polymer sheet 80 results in an enclosed surface channel such as the connecting channel 34 having a cross-section that resembles a rounded off rectangle or a semi-circle depending on how the channel was originally formed.

Turning again to FIG. 5, the connecting channel 34 provides fluid communication between the samples and/or reagents stored in the reservoirs 30 and the analytical portion of the cartridge housing 12 which houses the reaction channels 38 and the second valve 82. Samples traversing the connecting channel 34 enter the analytical portion of the cartridge housing 12 via a fluid portal 20 at the terminal end 35 of the connecting channel 34. The terminal end 35 of the connecting channel 34 is adjacent the second valve 82 and the fluid portal 20 between the two provides fluid communication between the connecting channel 34 and a second valve 82.

The second valve 82 is very similar to the first valve 40 in both construction and function. Accordingly, only the differences between the two are described here. The primary difference between the first valve 40 and the second valve 82 is that the second valve 82 is capable of controlling the flow of fluid through three or more fluid portals 20 simultaneously. The valve disk in the second valve (second valve disk 84) provides this functionality.

Turning now to FIGS. 1, 2B & C, and 4C, the second valve disk 84 is identical in construction to the first valve disk 44 with the exception of the second surface 88 of the second valve disk 84. (The second surface 88 of the second valve disk 84 is analogous to the second surface 56 of the first valve disk 44. The first surface 83 of the second valve disk is analogous to the first surface 54 of the first valve disk 44.) The second surface 88 of the second valve disk 84 is arranged in an “annular with central arm” pattern similar to that seen with the second surface 56 of the first valve disk 44.

The central arm 90 of the second disk second surface 88 contains a first channel 86 that is similar to the first channel 48 of the first valve disk 44. The first channel 86 of the second valve disk 84 is defined by a first end 85 and a second end 87. The first end 85 of the first channel 86 is proximate the center of the second valve disk 84 and the channel 86 extends toward the edge of the second valve disk 84. At the center of the second valve disk 84 the first channel 86 is in fluid communication with the fluid portal 20 that marks the start of the detecting channel 102. This fluid relationship between the second valve disk 84 and the detecting channel is shown in FIG. 2A. The detecting channel 102 carries fluid samples to a detector 126 and is discussed in more detail below.

The second valve disk 84 primarily differs from the first valve disk 44 in that the annular portion of the second surface 88 houses a second channel 92 that is capable of providing fluid communication between the inlets 104 and outlets 106 of one or more reaction channels 38 (FIG. 3).

More specifically, the second channel 92 is defined by an arcuate portion 91 and a radial portion 93 extending from one end of the arcuate portion 91. The arcuate portion 91 of the second channel 92 extends for distance along and within the arcuate portion of the second surface 88. The radial portion 93 extends from one end of the arcuate portion 91 toward the outer edge of the second valve disk 84 in a radial fashion as shown in FIG. 4C. The radial portion 93 of the second channel 92 is positioned proximate the distal (outer edge) portion of the second disk first channel 86 and is separated therefrom by a distance “B” (FIG. 4C) that is equal to the arcuate distance between the inlets 104 and the outlets 106 of the reaction channels 38 (FIG. 3).

It should also be noted that as with the first valve disk 44, the channels in the second valve disk 84 need not be housed within the raised platform created by the second surface 88 but can be formed on a flat first surface. However, for reasons stated previously, the use of a raised second surface 88 is preferred.

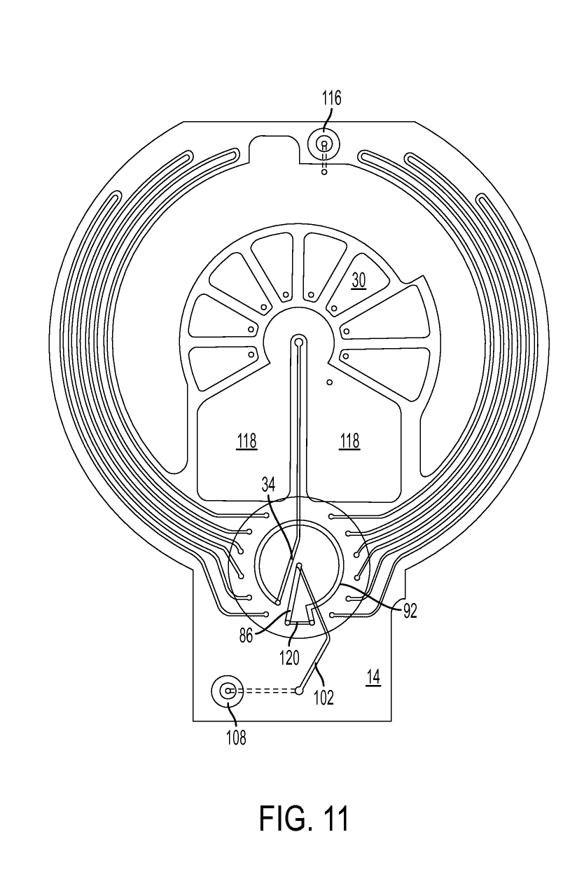

The configuration of the second channel 92 allows the second channel 92 to maintain continuous fluid communication with fluid portals 20 in 2 different but concentric circles. More specifically, and as shown in FIGS. 2B and 2C and 3, the outlet of the connecting channel 34 is positioned above the arcuate portion 91 of the second channel 92 while the inlets 104 and the outlets 106 of the reaction channels are positioned concentrically outside of the arcuate portion 91 of the second channel 92 (i.e., closer to the outer edge of the second valve disk 84). FIG. 11 shows the top surface 14 of a cartridge housing 12 having an alternative positioning of the connecting channel 34. FIG. 11 is discussed in more detail in the section of the detailed description addressing the bypass channel 120.

As the second valve disk 84 is rotated the radial portion 93 of the second channel 92 comes into intermittent and selective fluid communication with both the inlets 104 and the outlets 106 of the reaction channels while the arcuate portion 91 maintains fluid communication with the connecting channel 34. However, during use, the control system 100 is programmed such that the second valve disk 84 is rotated so as to only provide fluid communication between the radial portion 93 of the second channel 92 and the inlets 104 of each reaction channel 38.

During use the outlets 106 of the reaction channels 38 align with the second end 87 of the second disk first channel 86 due to the spacing “B” between the first channel 86 and the radial portion 93 of the second channel 92. As noted previously, the fluid portal 20 that defines the entrance of the detection channel 102 is located above the center of the second valve disk 84 and the first end 85 of the first channel 86 that traverses the center of the second valve disk 84. The placement of the detection channel entrance at the center of the second valve disk completes the fluid pathway that takes a fluid sample from a sample reservoir to a detection device 126, which is discussed in more detail in relation to an exemplary sample analysis.

The detection channel 102 terminates at a cartridge fluid exit portal 108 which is preferably integrated with a quick connect coupling that is suitable for mechanized and automatic alignment/connection to a fluid conduit 110 which is in turn connected to a pump 112. See FIG. 5.

The pump 112 used in the practice of the invention may be any pump capable of moving small quantities (e.g., milliliters, microliters) in a controlled and precise fashion. The pump should create a vacuum sufficient to draw small amounts of fluid from the reservoirs through the various channels and to the detector at flow rates suitable for the analytical study at issue.

There are several commercially available pumps that can be used in the practice of the invention. Peristaltic pumps such as 3200054, SP101, P625 commercially available from Dolomite, APT Instruments, and Instech, respectively, possess operational and control features that are suitable for use in the practice of the invention.

From the pump 112 the fluid from the cartridge flows to a waste receptacle 114. The waste receptacle can be a container apart from the cartridge 10 and connected to the pump by tubing. Alternatively, the cartridge 10 according to the invention can include its own built-in waste receptacle.

Turning again to FIGS. 1 and 3, the cartridge housing 12 shown therein contains an additional waste reservoir 114 formed in the same manner as the sample reservoirs 30. The waste reservoir 114 is not in fluid communication with the other fluid storage or fluid transfer components of the cartridge 10. A waste fluid inlet 116, preferably integrated with a quick connector which is in fluid communication with the pump 112, provides the means by which waste fluid flows into the waste reservoir 114.

The term quick connector, as used herein, means a device that is suitable for creating automated connections that establish fluid communication between the fluid exit portal 108 and the pump 112 and the waste fluid inlet portal 116 and the pump 112. For example, it is envisioned that the process will be automated such that once an operator places a cartridge within the overall system (discussed below) the fluid conduits need to establish fluid communication with the pump will move and lock into place automatically. The movement of arms or rotors to align the conduits with the exit 108 and inlet 116 portals will be controlled by the overall operating system. Similarly, the cartridge will contain guides (e.g., mechanical or magnetic) to ensure accurate alignment of the conduits with the portals.

In summary, the flow of a fluid sample through a cartridge 10 employing multiple sample reservoirs and multiple reaction channels is as follows (note: the passage through the various fluid portals 20 is only noted once but should be understood in the remainder of the steps): Reservoir 30→fluid portal 20→first channel 48 of first valve disk 44→connecting channel 34→arcuate portion 91 of second valve disk 84 second channel 92→radial portion 93 of second valve disk 84 second channel 92→reaction channel inlet 104→reaction channel 38→reaction channel outlet 106→second valve disk 84 first channel 86→detecting channel 102→exit portal 108. If an external pump 112 and internal waste reservoir 114 is utilized the flow continues from exit portal 108→pump 112→waste fluid inlet 116→waste reservoir 114.

Two technically optional but often required components that are anticipated as being part of most cartridges are a wash reservoir 118 and a bypass channel 120. As noted previously, one of the problems associated with known devices that test multiple samples and/or test for multiple analytes is cross contamination either by leaking or failure to clean the reaction channel between samples and/or failure to clean any channels or tubes that are common to multiple samples. The wash reservoir 118, the bypass channel 120 and the overall arrangement of components in the cartridge housing 12 addresses these problems.

Looking now at FIG. 1, the wash reservoir 118 is formed as a recess in the cartridge housing just as are the other reservoirs 30. The wash reservoir 118 is associated with a fluid portal 20 that establishes fluid communication with the first valve 40 first channel 48 just as are the other reservoirs. During operation the wash reservoir 118 contains a fluid that can be selectively pumped through the connecting channel 34 and then through the second valve 82 and the reaction channels 38 and the detecting channel 102 just as any other fluid in the reservoirs 30. The wash fluid may be any fluid (e.g., water, a detergent solution, a buffer solution) that is suitable for cleaning the aforementioned channels to eliminate or at least substantially reduce the potential for cross contamination between runs of different samples or different reagents.

It should also be noted that the circular/arcuate arrangement of the reservoirs 30 and wash reservoirs about a single point (i.e., the fluid portal 20 that marks the entrance to the connection channel 34) is a purposeful design innovation as is the arrangement of the reaction channels 38 about the second valve 82. Such a design shortens the length of the channels that are common to multiple samples which (1) reduces the amount of wash fluid required, and (2) reduces the likelihood of cross-contamination between samples.

The bypass channel 120 is another innovative feature of the cartridge 10. The bypass channel 120 is located in the portion of the housing 12 that encompassed by the second valve 82 as shown in FIGS. 1, 3 and 11. The bypass channel 120 is defined by an inlet and an outlet, both of which are associated with fluid portals 20 that are aligned in the same concentric circle as the inlets and outlets to the reaction channels 38. The bypass channel inlet and outlet engage with the second valve disk 82 first channel 86 and second channel 92 in the same manner as do the inlets and outlets of the reaction channels 38. Thus, when the second valve disk 82 is rotated such that the radial portion 93 of the second valve disk second channel 92 is in fluid communication with the bypass channel inlet the second valve disk first channel 86 is in fluid communication with the bypass channel outlet. This enables wash fluid to go from the connecting channel 34 directly to the detection channel 102 without having to go through a reaction channel 34 if that is desired. FIG. 11 illustrates the positioning of the second valve 82 when the bypass channel 120 is engaged.

Additional innovations that are incorporated into the cartridge 10 according to the invention relate to modifications to the housing 12 that improve analytical results. One such modification is a detector guide 127 that is situated proximate the detection channel 102. As shown in FIG. 2A, and as discussed in more detail below, the cartridge 10 is designed to present a sample to a detector for analysis. To do this in an automated fashion the cartridge 10 and the detector 126 must align in whatever fashion is needed for the detector 126 to complete its task. Additionally, the alignment must be repeated for each testing cycle and each cartridge. Misalignment between the detector 126 and the sample could cause, at a minimum, an error message and at worse provide incorrect data. The detector guide 127 is designed to couple with a matching guide on the detector 126 as the detector 126 moves into position (either manually or automatically) to read a sample to ensure correct placement of the detector 126. The detector guide 127 shown in FIG. 2A comprises two notches that would mate with guides extending from a detector. Other types of guides (e.g., magnetic based guides) can also be used.

Another such innovation is specific to optical detectors used in the practice of the invention. Although various types of detectors can be used in the practice of the invention, the detection channel 102 shown in the figures is based upon the exemplary immunoassay version of the cartridge according to the invention. As discussed in greater detail below, the analytical results that are provided in immunoassay tests are based in part on an optical reader detecting and quantifying a colorimetric change in a fluid sample as it passes by the detector. The detection channel 102 shown in the figures is similar to the other channels in that it has a vertical portion (a fluid portal) that traverses the body of the cartridge housing 12 which is best shown in FIG. 2A. This vertical portion (fluid portal) within the detection channel 102 is the optical window 132 for the optical detector 126.

Generally speaking, an optical detector comprises a light source on one side and a light sensor directly opposed to the light source. As a sample moves between the source and the sensor the sensor detects variations in the amount of light transmitted through the sample (or absorbed by the sample) versus the amount of light emitted by the source. This variation is then processed using a series of algorithms to obtain information about the sample.

In preferred embodiments the cartridge housing 12 is made of a transparent material which facilitates subjective analysis of whether or not the cartridge is functioning correctly (e.g., a technician can view fluid as it passes through the cartridge). When an optical detector is used in the practice of the invention the light source is placed directly across from one end of the optical window 132 and the light sensor is placed directly across from the other end. To ensure that adequate light is available many optical sensors are designed to provide more light than necessary to conduct a successful analysis. When this light is shown through a transparent housing, some of the light may traverse the portion of the housing immediately adjacent the optical window 132, not be altered by any absorbance by the sample, yet still be read by the optical reader. The reading of such “unaltered” light can skew analytical readings.

This issue can be addressed in multiple ways in the practice of the invention. In one embodiment an opaque polymer layer 134 (FIG. 1) was placed above and below the location of the optical window 132 prior to placement of the polymer sheet 80 and the forming of the optical window 132 in the housing. This opaque material block light from traversing the housing outside of the optical window 132. Prototypes of the cartridge according to the invention used a thin opaque polycarbonate film to create the opaque layer 134 but other materials can be used as well.

Another method utilized to address the issue of light leakage around the optical window is to place an opaque sleeve or tube (not shown) within the optical window 132 combined with an aperture on the optical detector that is aligned with the opaque sleeve.

In addition to the above described cartridge 10, the invention also encompasses a system 124 for detecting the presence of an analyte in a fluid sample. The primary components of the system according to the invention are the above described cartridge 10, a detector 126 to detect the presence an analyte in a fluid sample, a power source 122, an electronic control system/microprocessor 100, a drive mechanism 70 such as that previously discussed, and a pump 112 such as that previously described. The system according to the invention is schematically represented in FIG. 6.

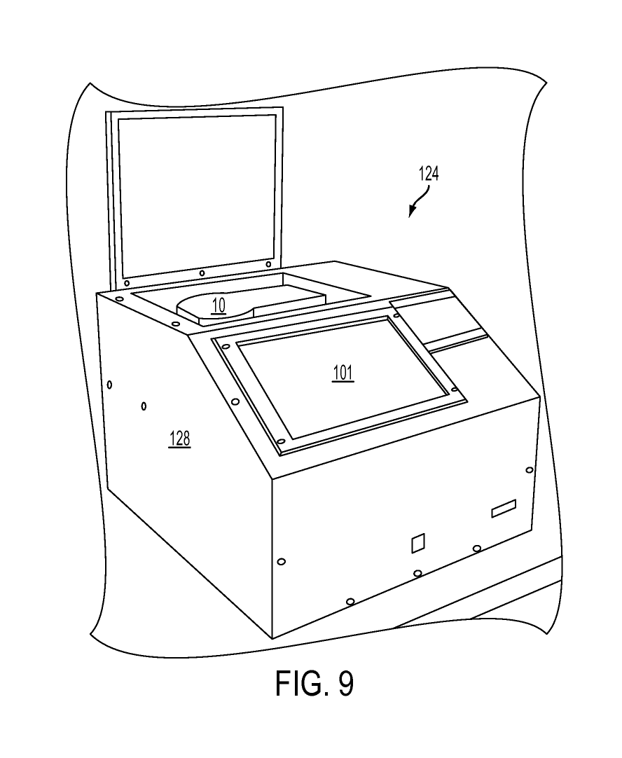

In preferred embodiments of the system according to the invention the above enumerated components (with the exception of the cartridges) are packaged into a single unit 124 such as that shown in FIG. 9 that receives and analyzes cartridges 10. For example, the system could include a suitable housing 128 that fixably houses the frame 79 from which the motors 76 are suspended. The frame 79 would also include suitable attaching means 130 (e.g., snap fit connectors) that would receive a cartridge 10 and help guide the disk support spindles 72 to receive the drive shafts 78 of the motors 76.

The detector 126 is then placed proximate the detection channel 102 and one or more samples are analyzed. Preferably, and as schematically shown in FIG. 6, the detector 126 is automatically moved into (and out of) position by an automated arm 129 (or some other positioning device) that is controlled by the control system/microprocessor. The movement of such a positioning system is illustrated by the dotted lines in FIG. 6.

The drive mechanism 70 (e.g., motors 76 and drive shafts 78) are connected to a controlling system 100 which consists of a programmable microprocessor 136 such as those commonly used to control scientific instruments. The controlling system 100 is preferably connected to a touch pad controller 101 as is common in the art (FIG. 9).

During use a technician need only insert a new cartridge 10 containing fresh samples onto the frame 79 until it is properly positioned. The technician then follows the pre-programmed instructions on the touch pad controller 101 which then initiates an analysis sequence. The microprocessor 136 controls the drive mechanism 70 which rotates the valves to the appropriate position to establish the appropriate fluid communication. The pump 112 is activated to draw sample, reagent, or wash through the cartridge, past the detector, and into the waste receptacle. The process is repeated for each sample in the cartridge.

Turning now to the more analytical aspects of the invention, and as noted in the background section, one purpose of the cartridge according to the invention is to detect the presence of certain analytes in a sample. Preferably, the invention provides a detection system that is capable of quantifying the amount of analyte in a sample as well. In many embodiments of the invention this detection/analysis will take place in one or more reaction channels 38 that are present in the cartridge 10 and by a detector adjacent to the cartridge.

The reaction channels 38 are capillary channels very similar to the connecting channel 34 with respect to construction and cross-section. It should be noted that in some embodiments of the invention that are designed to be “single sample” cartridges, the cartridge could have only one reaction channel 38 or the connecting channel 34 and/or the detecting channel 102 can be modified to incorporate features of the reaction channels 38. The necessary modifications to make this adjustment will be readily apparent to those skilled in the art.

However, in preferred embodiments, the cartridge 10 according to the invention is designed to accommodate multiple samples and detect multiple analytes. Accordingly, in preferred embodiments the cartridge according to the invention will contain multiple reaction channels 38.

As shown in FIG. 2, the exemplary cartridge housing 12 contains a plurality of reaction channels 38. The length of the reaction channels 38 may vary depending upon the analytical process that is conducted therein (e.g., some detection processes may require longer residence times).

Each reaction channel 38 is defined by a lumen 92 where the thin polymer sheet forms the “top” of the lumen and the milled or molded recess in the cartridge surface forms a lumen surface 94. Attached to at least a portion of said lumen surface 94 are detection components 96 suitable for aiding in the detection of an analyte in a fluid sample. The term “attached” as used herein means physically attached to or incorporated into the lumen surface by chemical, mechanical, electrostatic or any other suitable means and also includes simple coating technologies such as allowing a fluid containing a detection component to dry on the lumen surface. The detection component 96 can be anything that can be attached to the lumen surface that aids in the detection of an analyte in a fluid sample. The entire range of possible detection components 96 that can be used in the practice of the invention is too great for detailed analysis but those skilled in the art will recognize that such detection components include but are not limited to biological compounds (e.g., antibodies), inorganic compounds, elements of the periodic table, and organic compounds and combinations or any or all of the above. Suitable biological compounds include but are not limited to cells, parts of cells, bacteria, parts of bacteria, viruses, nucleotides, nucleic acids (e.g., DNA, RNA, mRNA, miRNA, rRNA), proteins, peptides, amino acids, antibodies, antigens, haptens, monosaccharides, polysaccharides, lipids, and combinations of any or all of the above.

As noted previously, the cartridges according to the invention are well suited for use with immunoassay technology and that technology is utilized to as a narrative convenience to aid in the description of the invention. However, this narrative tool should not be interpreted as narrowing the scope of the invention.

The next several paragraphs describe the preparation of a cartridge for testing an analyte. Escherichia. coli 0157:H7 is used as the exemplary analyte. It is envisioned that the cartridges used in the practice of the invention will be supplied pre-packaged and pre-loaded with the reagents, detection components and wash fluids necessary to complete the necessary number of sample tests.

Generally speaking, the reaction channel 38 lumen surface 94 is modified by having a detection component 96 such as a capture antibody (e.g., anti-E. coli 0157:H7) attached thereto as shown schematically in FIG. 7. The techniques for attaching such antibodies to a polymer surface (e.g., physical absorption or chemical covalent bonding) are known in the art and need not be discussed in detail herein.

One of the larger reservoirs 30 (reagent reservoir) is pre-loaded with an alkaline phosphatase-labeled anti-E. coli 0157:H7 antibody. Another large reservoir 30 (reagent reservoir) is pre-loaded with alkaline phosphatase substrate. The wash reservoir 118 is pre-loaded with the appropriate wash.

For purposes of this example two fluid samples are placed in two of the smaller sample reservoirs 30 and the cartridge 10 is placed in the system 124 and the system is activated. The valves are adjusted to send a sample through the cartridge. If there is E. coli (i.e., the analyte) in the sample it should be captured by the detection components 96 (e.g., anti-E. coli antibodies). The captured E. coli in FIG. 7 is identified as element 97. It is to be understood that the microprocessor adjusts the valves and conducts a washing step as needed during the course of a sample analysis.

The valves are adjusted again to align with the reagent reservoir containing the alkaline phosphatase-labeled anti-E. coli 0157:H7 antibody which then attaches to the captured E. coli from the sample (element 98 of FIG. 7).

Lastly, the reagent containing the alkaline phosphatase substrate is pulled through the system where it reacts with the labeled antibody and creates a color change in the fluid moving through the system. The color change provides a qualitative indication of the presence of E. coli which may suffice for some applications. However, the fluid is analyzed by an optical detector 126 for optical absorbance as it passes through the optical window 132 and the microprocessor runs the appropriate algorithms to develop a quantitative assessment of the quantity of E. coli in the sample.

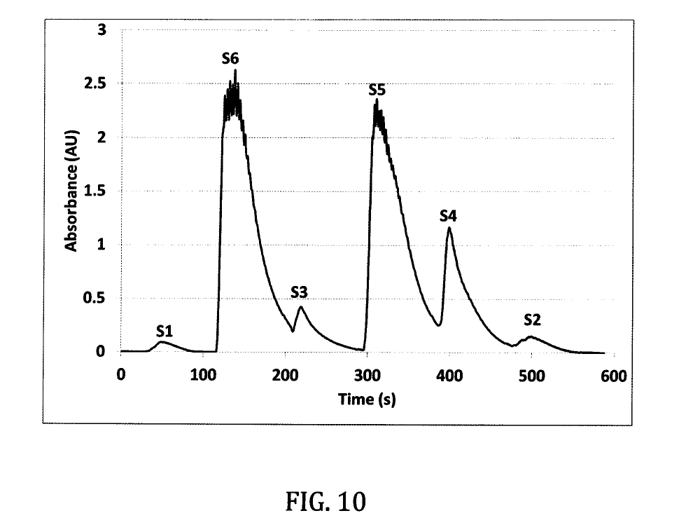

FIG. 10 provides the results for detection of Escherichia coli 0157:H7 in a plurality of samples using the cartridge-based system. The concentrations of E. coli 0157:H7 in the samples were: S1, 0 CFU/mL (blank); S2, 0 CFU/mL (blank); S3, 5×10³ CFU/mL; S4, 2.5×10⁴ CFU/mL; S5, 1.25×10⁵ CFU/mL; S6, 6.25×10⁵ CFU/mL.

A still further aspect of the invention is a method to detect an analyte in a fluid sample. Many steps in the method according to the invention have been discussed above and this description incorporates all of the previous detail regarding the construction and function of the cartridges and systems of the invention.

In a basic form, the method according to the invention is a method of detecting an analyte in a fluid sample, using a cartridge 10. The method comprises the steps of providing a cartridge 10 prepared to receive a sample, placing a first sample within the cartridge 10, and causing the first sample to pass through a first reaction channel 38, and detecting any analyte in the first sample. In preferred embodiments of the method, the method further comprises the steps of placing a second sample within the cartridge, causing the second sample to pass through a second reaction channel, and detecting any analyte in the second sample. For methods that incorporate the testing of multiple samples the method includes passing a wash fluid through the common channels (e.g., the connecting channel, the detection channel) of the cartridge in between the first and second samples (and any additional samples) as needed.

More specifically and when taken in light of the cartridge 10 architecture, the method according to the invention comprises the steps of placing a sample in a first reservoir 30; aligning a first valve 40 to establish fluid communication between the reservoir 30 and a connecting channel 34; aligning a second valve 82 to establish fluid communication between the connecting channel 34, the inlet of a reaction channel 104, the outlet of a reaction channel 106, and an analyte detection channel 102 to thereby establish fluid communication between the reservoir 30 and the analyte detection channel 102; drawing a sample from the first reservoir 30 through the reaction channel 34 and through the detection channel 102; and detecting the presence of an analyte in the sample as said sample passes through the analyte detection channel 102.

In preferred embodiments of the more cartridge-centric view of the method according to the invention, the method comprises the additional steps of placing a second sample in a second reservoir 30; drawing the second sample through a second reaction channel 34 and through the detection channel 102; and drawing a wash fluid through at least the detection channel 102 (and preferably through all common channels if needed) in between the analysis of the first and sample samples.

In preferred embodiments the reaction channels 34 used in the methods according to the invention are prepared such that the methods are suited for use in an immunoassay detection analysis.

While the invention is described with respect to various embodiments thereof, it will be understood by those skilled in the art that various changes in detail may be made therein without departing from the spirit, scope, and teaching of the invention. Accordingly, the invention herein disclosed is to be limited only as specified in the claims.

That which is claimed is:

1. A disk for controlling the flow of fluid through a cartridge housing, said disk comprising a circular first face having a first channel, said first channel suitable for establishing fluid communication between a first fluid portal and a second fluid portal of said cartridge housing.

2. A disk according to claim 1 wherein said circular first face is further defined by a first surface and a second surface, said second surface apart from and parallel to said first surface, said first channel located within said second surface, said first channel extending from the center of said circular first face to a point distal to said center.

3. A disk according to claim 1 wherein said disk comprises a polymer, said polymer suitable for forming a fluid tight seal with said cartridge housing and having a coefficient of friction sufficient to allow lubricant free rotation against said cartridge housing.

4. A disk according to claim 1 wherein the hardness of said disk polymer is less than the hardness of said cartridge housing.

5. A disk according to claim 1 further comprising a disk support supporting said disk, said disk support having a channel for receiving a drive mechanism.

6. A disk according to claim 1 wherein said disk is integral with said disk support.

7. A disk according to claim 1 wherein said circular first face has a second channel, said second channel having an arcuate portion and a radial portion, said second channel suitable for establishing fluid communication between two fluid portals of said cartridge housing.

8. A disk according to claim 7 wherein said circular first face is further defined by a first surface and a second surface, said second surface apart from and parallel to said first surface, said first channel located within said second surface, said first channel extending from the center of said circular first face to a point distal to said center, said second channel located within said second surface.

9. A disk according to claim 7 wherein said two fluid portals in fluid communication with said second channel are located on two different but concentric circles.

10. A cartridge for use in detecting an analyte comprising the disk of claim 5, said cartridge further comprising
a cartridge housing;
a first sample reservoir integral with said cartridge housing, said first sample reservoir in fluid communication said first channel of said disk;
a connecting channel integral with said cartridge housing, said connecting channel in fluid communication with said first channel;
a reaction channel integral with said cartridge housing, said reaction channel in fluid communication with said connecting channel;
a detection channel in fluid communication with said reaction channel; and
a fluid exit portal.

11. A cartridge according to claim 10 wherein said reaction channel is defined by a lumen having a lumen surface wherein at least a portion of said lumen surface comprises at least one detection component attached thereto.

12. A cartridge according to claim 11 wherein said detection component is suitable for aiding in the detection of at least one analyte in a sample, said at least one analyte selected from the group consisting of biological compounds, inorganic compounds, elements of the periodic table, organic compounds, and combinations thereof.

13. A cartridge according to claim 12 wherein said biological compounds are selected from the group consisting of cells, parts of cells, bacteria, parts of bacteria, viruses, nucleotides, nucleic acids, RNA, DNA, proteins, peptides, amino acids, antibodies, antigens, haptens, monosaccharides, polysaccharides, lipids, and combinations thereof.

14. A cartridge according to claim 10 further comprising a fluid inlet portal integral with said cartridge housing, said fluid inlet portal in fluid communication with a waste reservoir.

15. A cartridge according to claim 10 further comprising:
a plurality of sample reservoirs capable of fluid communication with said first channel, and
a plurality of reaction channels capable of fluid communication with said connecting channel.

16. A system for detecting the presence of an analyte in a fluid sample, the system comprising:
the cartridge according to claim 15;
a pump in fluid communication with said fluid exit portal;
a detector for detecting the presence of an analyte in a fluid in said detection channel; and
a control system for controlling said pump and said detector.

17. A cartridge for use in detecting an analyte in a fluid sample, said cartridge comprising:
a cartridge housing;
a reservoir integral with said cartridge housing;
a connecting channel integral with said cartridge housing;
a first valve capable of establishing fluid communication between said reservoir and said connecting channel;
a reaction channel having an inlet and an outlet, said reaction channel integral with said cartridge housing;
a detection channel integral with said cartridge housing; and
a second valve capable of establishing fluid communication (1) between said connecting channel and said reaction channel, and (2) between said reaction channel and said detection channel.

18. A cartridge according to claim 17 wherein said first valve comprises:
a valve housing;
a first valve disk received by said valve housing, said first valve disk having a first channel, said first channel capable of establishing fluid communication between said first reservoir and said connecting channel.

19. A cartridge according to claim 18 further comprising a plurality of reservoirs wherein said first channel is capable of establishing fluid communication between each of said plurality of reservoirs and said connecting channel.

20. A cartridge according to claim 19 wherein said first valve disk comprises a polymer, said polymer suitable for forming a fluid tight seal with said cartridge housing and having a coefficient of friction sufficient to allow lubricant free rotation against said cartridge housing.

21. A cartridge according to claim 20 wherein said first valve further comprises a biasing mechanism for maintaining said circular first face adjacent said cartridge housing.

22. A cartridge according to claim 19 wherein said first valve further comprises a disk support which supports said first valve disk, said disk support having a channel for engaging with a drive mechanism.

23. A cartridge according to claim 22 wherein said first valve disk is integrated with said disk support.

24. A cartridge according to claim 19 further comprising a plurality of reaction channels wherein said second valve is capable of establishing fluid communication between each of said plurality of reaction channels and said connecting channel.

25. A cartridge according to claim 24 wherein said second valve comprises:
a valve housing;
a second valve disk received by said valve housing, said second valve disk having a first channel and a second channel, wherein said first channel is capable of establishing fluid communication between each of said plurality of reaction channels and said detection channel, and wherein said second channel is capable of establishing fluid communication between said connecting channel and each of said plurality of reaction channels.

26. A cartridge according to claim 25 wherein said second valve disk comprises a polymer, said polymer suitable for forming a fluid tight seal with said cartridge housing and having a coefficient of friction sufficient to allow lubricant free rotation against said cartridge housing.

27. A cartridge according to claim 25 wherein said second valve further comprises a disk support which supports said second valve disk, said disk support having a channel for engaging with a drive mechanism.

28. A cartridge according to claim 27 wherein said second valve disk is integrated with said disk support.

29. A cartridge according to claim 25 further comprising a bypass channel integral with said cartridge housing, wherein said second valve is capable of establishing fluid communication between said connection channel, said bypass channel, and said detection channel.

30. A cartridge according to claim 6 wherein said reaction channel is defined by a lumen and a lumen surface, at least a portion of said lumen surface having a detection component attached thereto and wherein said detection component is suitable for aiding in the detection of an analyte in a fluid sample.

31. A cartridge according to claim 30 wherein said detection component is suitable for aiding in the detection of an analyte in a fluid sample, said analyte selected from the group consisting of biological compounds, inorganic compounds, elements of the periodic table, and organic compounds.

32. A cartridge according to claim 24 further comprising a polymer film overlying said reservoirs and said channels.

33. A system for detecting an analyte in a fluid sample, said system comprising:
a cartridge according to claim 24;
a drive mechanism engaging said first and second valves;
a detector for detecting an analyte; and
a pump, said pump in fluid communication with said detection channel.

34. A system according to claim 33 wherein said detector is an optical detector.

35. A system according to claim 33 wherein said drive mechanism comprises a drive shaft and a motor, said motor being suspended from a frame.

36. A system according to claim 33 further comprising a control system, said control system connected to said pump, said drive mechanism, and said detector.

37. A method of detecting an analyte in a fluid sample, using the cartridge of claim 17 comprising the steps of:
providing a cartridge prepared to receive a sample;
placing a first sample within said cartridge;
causing said first sample to pass through a first reaction channel; and
detecting any analyte in said first sample.

38. A method according to claim 37 further comprising the steps of:
placing a second sample within said cartridge;
causing said second sample to pass through a second reaction channel; and
detecting any analyte in said second sample.

39. A method according to claim 38 further comprising the step of passing a wash fluid through said detection channel in between said first and second sample.

40. The method of claim 37 wherein said reaction channel is defined by a lumen and a lumen surface, at least a portion of said lumen surface having a detection component attached thereto and wherein said detection component is suitable for aiding in the detection of an analyte in a fluid sample.

41. The method of claim 40 wherein said detection component is suitable for aiding in the detection of an analyte in a fluid sample, said analyte selected from the group consisting of biological compounds, inorganic compounds, elements of the periodic table, and organic compounds.

42. A method for detecting an analyte in a fluid sample, the method comprising the steps of:
placing a sample in a first reservoir;
aligning a first valve to establish fluid communication between said reservoir and a connecting channel;
aligning a second valve to establish fluid communication between said connecting channel and the inlet of a reaction channel and the outlet of said reaction channel and an analyte detection channel to thereby establish fluid communication between said reservoir and said analyte detection channel;
drawing a sample from said first reservoir through said reaction channel and through said detection channel; and
detecting the presence of an analyte in the sample as said sample passes through said analyte detection channel.

43. A method according to claim 42 further comprising the steps of:
placing a second sample in a second reservoir;
drawing said second sample through a second reaction channel and through said detection channel; and
drawing a wash fluid through at least said detection channel in between said first and sample samples.

44. A method according to claim 42 wherein said reaction channel is defined by a lumen and a lumen surface, at least a portion of said lumen surface having a detection component attached thereto and wherein said detection component is suitable for aiding in the detection of an analyte in a fluid sample.

45. A method according to claim 44 wherein said detection component is suitable for aiding in the detection of an analyte in a fluid sample, said analyte selected from the group consisting of biological compounds, inorganic compounds, elements of the periodic table, and organic compounds.

46. A method according to claim 44 wherein said detection component is suitable for use in an immunoassay detection technique.
